(19) United States
(12) Patent Application Publication
(10) Pub. No.: US 2023/0405166 A1
(43) Pub. Date: Dec. 21, 2023

(54) DEVICE FOR DECONTAMINATING A HOLLOW OBJECT DEFINING AN INTERNAL CAVITY, AND ASSOCIATED DISPENSING MACHINE AND METHOD

(71) Applicant: CAPSUM, MARSEILLE (FR)

(72) Inventors: Yan eric PAFUMI, GARDANNE (FR); Mathieu GOUTAYER, SAINT MALO (FR)

(21) Appl. No.: 18/251,833

(22) PCT Filed: Nov. 8, 2021
(86) PCT No.: PCT/EP2021/080862

§ 371 Date: May 4, 2023

(30) Foreign Application Priority Data
Nov. 9, 2020 (FR) 2011483

(51) Int. Cl. A61L 2/10; B65B 55/08; B65B 65/08; B65B 39/00

(52) U.S. Cl. CPC A61L 2/10; B65B 55/08; B65B 65/08; B65B 39/007; B65B 2210/06; A61L 2202/11; A61L 2202/14; A61L 2202/122

(57) ABSTRACT

The decontamination device includes a protective enclosure defining an inner space containing at least a portion of a decontamination member and a holder for the hollow object at least in a decontamination position. The enclosure defines an access passage to the inner space and at least one wall for closing the access passage, the closure wall and the access passage being movable the one relative to the other between an open configuration of the access passage which the closure wall is able to occupy in a rest position of the decontamination member and a closed configuration of the access passage which the closure wall occupies in the decontamination position of the decontamination member.

The present invention relates to an device for decontaminating a hollow object defining an internal cavity, including:
a decontamination unit, the decontamination unit including a holder intended to receive and position the hollow object, a decontamination member intended to be introduced into the internal cavity, and a mechanism for the relative displacement of the decontamination member relative to the holder able to displace the decontamination member and the holder relatively the one in relation to the other between a rest position intended for positioning the hollow object in the holder and a decontamination position, in which the decontamination member is intended to be inserted into the internal cavity of the hollow object received in the holder
a control unit able to activate the decontamination member in the decontamination position.

The decontamination device is intended to be placed in particular in a commercial space, to form a self-contained device, advantageously associated with a separate machine for dispensing bulk product into the hollow object. Alternatively, the decontamination device is integrated into a machine for dispensing bulk material into the hollow object.

The product intended to be dispensed into the hollow object in is for example a food product, a cleaning product (in particular a detergent), or a cosmetic product. Preferably, the product is a cosmetic product.

Before industrialization, bulk and deposit had their place in all shops. This mode of distribution declined with the development of large stores, working women and the explosion of the packaging industry. Since the “glorious thirties”, packaging has evolved considerably and the layers of packaging have multiplied. Today, for economic and ecological reasons, more virtuous modes of consumption and distribution are reappearing and reinventing themselves.

In particular, we can observe a return in stores of bulk distribution machines. These machines are adapted to all types of compositions, liquid or solid, notably food, detergents, and even cosmetics. By way of illustration, the machines for distribution of liquid bulk in store are proposed by 3J Development (3JD) or CoZie. The machine described in the French patent application registered under the number FR2008422 can also be mentioned.

However, the deployment of this type of machine is hampered by sanitary and regulatory issues, as this mode of consumption and distribution relies on the reuse of packaging. This reuse implies guaranteeing satisfactory cleanliness and sterility of the packaging before it is filled, otherwise it could contaminate, or even endanger, consumers.

This generally obliges stores to implement logistics to overcome this difficulty, either by decontaminating the packaging directly in the store, which is generally not feasible due to a lack of space and/or the absence of a water point in the store, or by exchanging it with new or previously decontaminated packaging, which necessarily implies the use of an industry responsible for collecting the used packaging in the store, decontaminating it and returning it to the store.

However, these solutions are not entirely satisfactory. Stores do not always have sufficient space to store used packaging brought back by consumers. Furthermore, these logistics are clearly not in line with the objectives sought in terms of economy and ecology of bulk distribution machines in stores. They are also costly.

It is therefore very difficult, if not impossible, today, to propose a fast, easy to use, safe, economic and ecological decontamination solution for empty packaging brought back by consumers in stores. This limits, or even prevents, the deployment of this new mode of consumption despite its ecological and economic advantages.

There are already industrial decontamination installations, in particular with light sources, adapted to the mass production of objects presenting a cavity. As an example, the decontamination devices described in WO2010012915 and EP2816002 can be mentioned.

However, these decontamination devices are designed for the mass production industry and operate continuously. They are therefore not suitable for one-off use in a store, that is, by an operator or even the consumer, particularly in terms of size, flexibility of use and above all safety.

One aim of the invention is to provide a device for decontaminating hollow objects, which is suitable for occasional handling in a store by an operator, or even by the consumer, in particular in terms of dimensions, flexibility of use and safety, in order to contribute to the deployment of bulk distribution machines, in particular for food, detergent or cosmetic compositions.

To this end, the invention has as its object a device of the aforementioned type, characterized by a protective enclosure defining an inner space containing at least a portion of the decontamination member and the holder at least in the decontamination position, the enclosure defining a passage for access to the inner space, and at least one wall for closing the access passage, the closure wall and the access passage being movable relative to each other, between an open configuration of the access passage which the closure wall is able to occupy in the rest position of the decontamination member, and a closed configuration of the access passage which the closure wall occupies in the decontamination position of the decontamination member.

The device according to the invention may comprise one or more of the following features, taken alone or in any technically possible combination:
the mechanism for relative displacement of the decontamination member relative to the holder is able to displace the decontamination member relative to the holder between the rest position and the decontamination position, the holder being static, or is able to displace the holder relative to the decontamination member between the rest position and the decontamination position, the decontamination member being static, or is able to simultaneously displace the decontamination member and the holder between the rest position and the decontamination position;
the closure wall is movable relative to the access passage between the open configuration and the closed configuration, the access passage being static, or the access passage is movable relative to the closure wall between the open configuration and the closed configuration, the closure wall being static, or the access passage and the closure wall are movable simultaneously between the open configuration and the closed configuration;
the inner space of the enclosure contains the decontamination unit at least in the decontamination position;
the inner space of the enclosure comprises a holder access region communicating with the access passage, and a technical region, advantageously separated from the access region by at least one partition,
the decontamination member is totally contained in the technical region in the rest position, and is at least partially inserted in the access region in the decontamination position;
the access region and the access passage define an alcove in the enclosure;
the alcove contains the holder, at least in the rest position;
the closure wall occupies the open configuration in the rest position;
the relative displacement of the decontamination member relative to the access passage from the rest position to the decontamination position causes the relative displacement of the closure wall relative to the holder from the open configuration to the closed configuration
and/or the relative displacement of the closure wall relative to the access passage from the open configuration to the closed configuration causes the relative displacement of the decontamination member relative to the holder from the rest position to the decontamination position,
the relative displacement of the decontamination member relative to the holder and the relative displacement of the closure wall relative to the access passage are preferably subject to each other;
in the rest position, the closure wall is movable relative to the access passage between the open configuration and the closed configuration;
the closure wall is held at rest in the closed configuration;
the control unit is able to control the relative displacement of the decontamination member relative to the holder between the rest position and the decontamination position and/or is able to control the relative displacement of the closure wall relative to the access passage from the open configuration to the closed configuration;
it comprises a first sensor for detecting the positioning of the hollow object in the holder and/or a second sensor for detecting the closed configuration of the closure wall, and/or a third sensor for detecting the decontamination position, the control unit being able to activate the decontamination member when the first sensor detects that the object is placed in the holder and/or when the second sensor detects that the closure wall is in the closed configuration, and/or when the third sensor detects that the decontamination member is in the decontamination position;
the closure wall is a panel that is movable in translation or rotation relative to the access passage or the enclosure comprises a movable drawer defining the access passage, the drawer being movable relative to the closure wall;
the holder includes a region for guiding and/or receiving at least part of the hollow object, in particular a neck of the hollow object, able to fix the position and alignment of the hollow object in the holder, the guiding and/or receiving region being in particular a sleeve complementary to a neck of the hollow object, a surface for guiding a neck of the hollow object by gravity or an indentation for receiving the hollow object, which is complementary in shape to the hollow object, is able to fix the position and alignment of the hollow object in the holder;
the holder includes a flap for holding the hollow object in the guiding and/or receiving region, which can be moved, in particular by pivoting, between a position for placing the hollow object in the guiding and/or receiving region and a position for holding the hollow object in the guiding and/or receiving region;
the decontamination member includes a light source, in particular a continuous or pulsed UV light emission source;
the holder is arranged above the decontamination member in the rest position.

The invention also has as its object a machine for dispensing bulk product into a hollow object defining an internal cavity, the machine comprising at least one product reservoir, at least one dispensing nozzle intended to be placed facing the internal cavity of the hollow object, and optionally an assembly for measuring the quantity of product introduced into the internal cavity of the hollow object, the machine further including a decontamination device as defined above.

The invention also has as its object a method for decontaminating a hollow object presenting an internal cavity comprising the following steps:
providing a decontamination device as defined above;
implementation of a decontamination sequence comprising the following steps:
the decontamination member being in its rest position and the closure wall being in its open configuration, introduction of a hollow object presenting an internal cavity into the holder;
transition of the closure wall to the closed configuration and transition of the decontamination member to the decontamination position, the decontamination member is inserted into the internal cavity;
activation of the decontamination member by the control unit; and, at the end of the decontamination sequence:
transition of the closure wall into the open configuration and transition of the decontamination member into the rest position, the decontamination member exiting the internal cavity;
removing the hollow object from the holder.

The device according to the invention may comprise one or more of the following features, taken alone or in any technically possible combination:
after removal of the hollow object from the holder, the control unit controls the transition of the closure wall into the closed configuration and/or the transition of the displacement member from the rest position to the decontamination position, without activating the decontamination member;
it then includes the following steps:
   providing a new hollow object to be decontaminated;
   transition of the decontamination member to its rest position and/or the closure wall to its open configuration,
   implementation of a new decontamination sequence comprising the following steps:
introduction of the new hollow object presenting an internal cavity into the holder,
transition of the closure wall to the closed configuration and transition of the decontamination member to its decontamination position, the decontamination member being inserted into the internal cavity of the new hollow object;
activation of the decontamination member by the control unit.

The invention will be better understood upon reading the following description, given only by way of example, and made with reference to the attached drawings, in which:

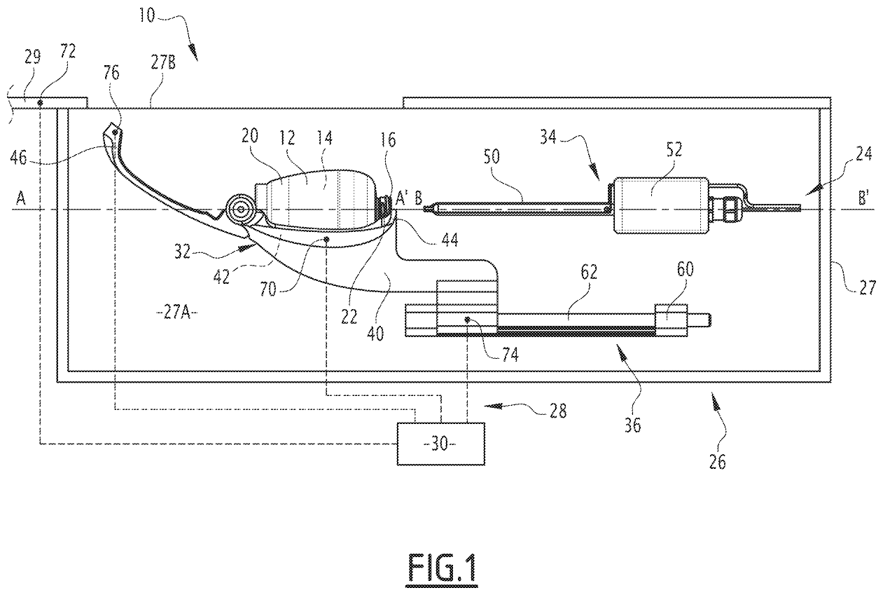

FIG. 1 is a schematic view, taken in partial section, of a first decontamination device according to the invention, in an open configuration allowing the loading of a hollow object;

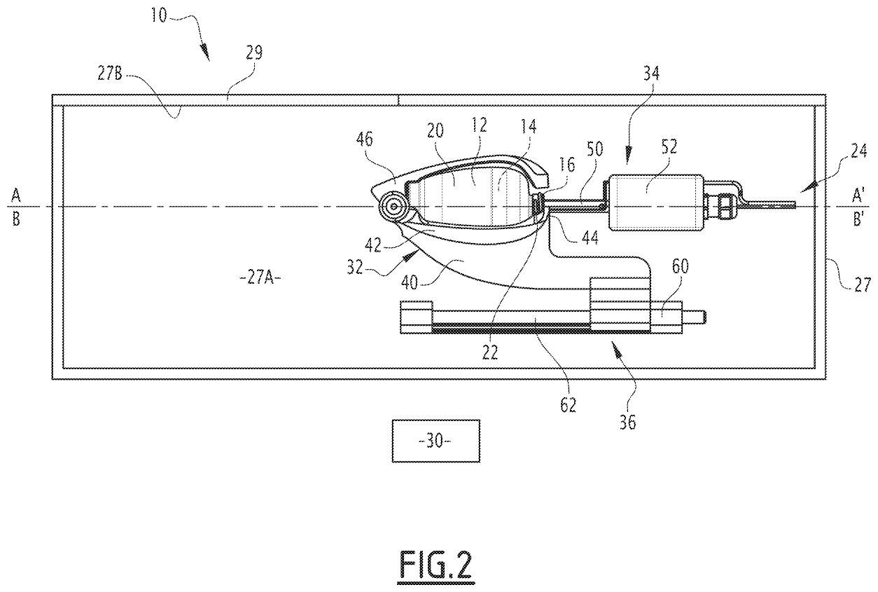

FIG. 2 is a similar view to FIG. 1, in a closed configuration in which decontamination is implemented;

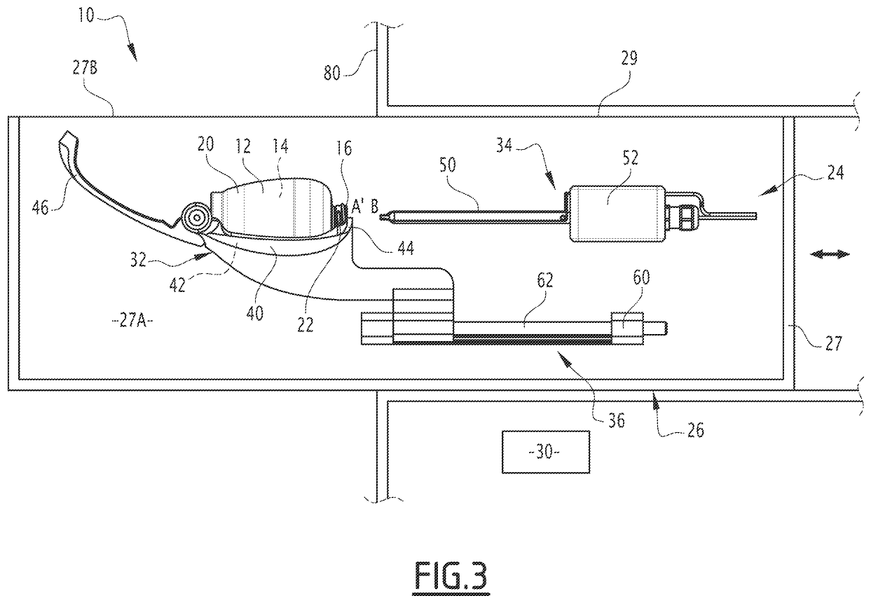

FIG. 3 is a view similar to FIG. 1 of one alternative of the device according to the invention;

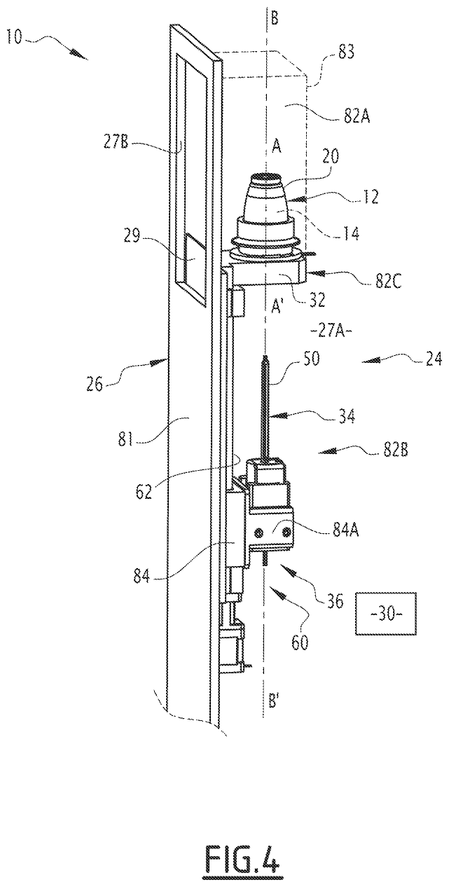

FIG. 4 is a view similar to FIG. 1 of another alternative of the device according to the invention;

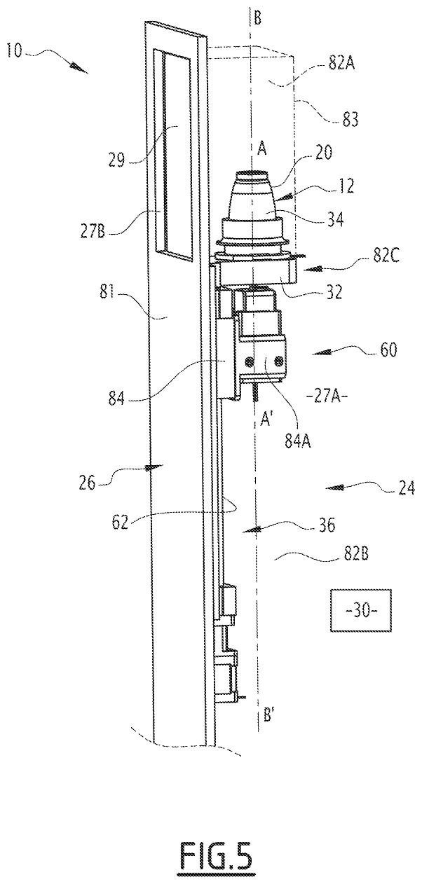

FIG. 5 is a view similar to FIG. 2 of the device according to the invention shown in FIG. 4;

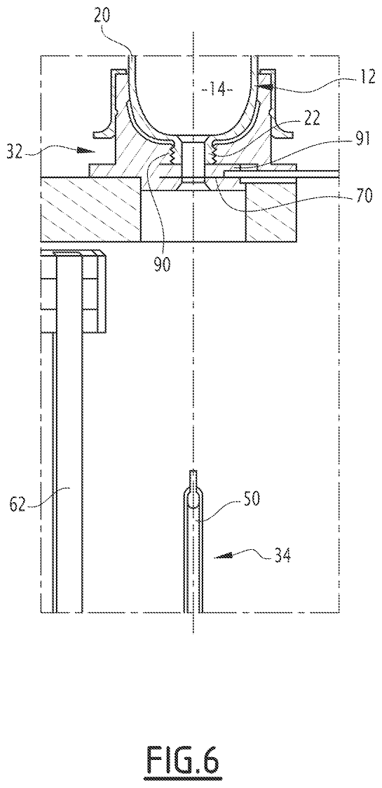

FIG. 6 is a schematically illustrative view of the holder of a hollow object applicable to the device according to the invention shown in FIG. 4;

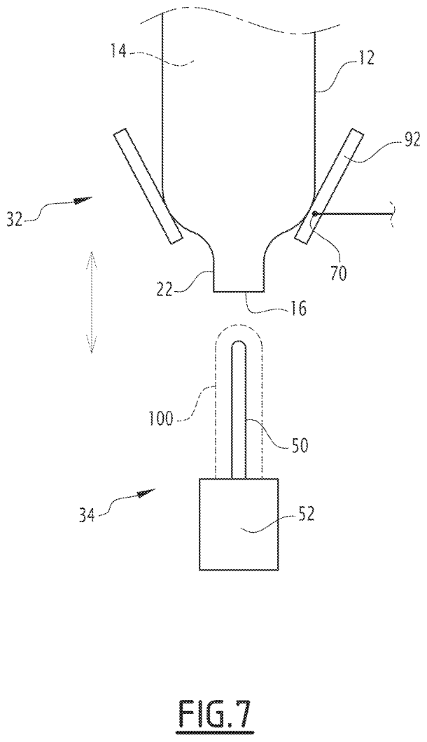

FIG. 7 is a view similar to FIG. 6, of another alternative of the device according to the invention;

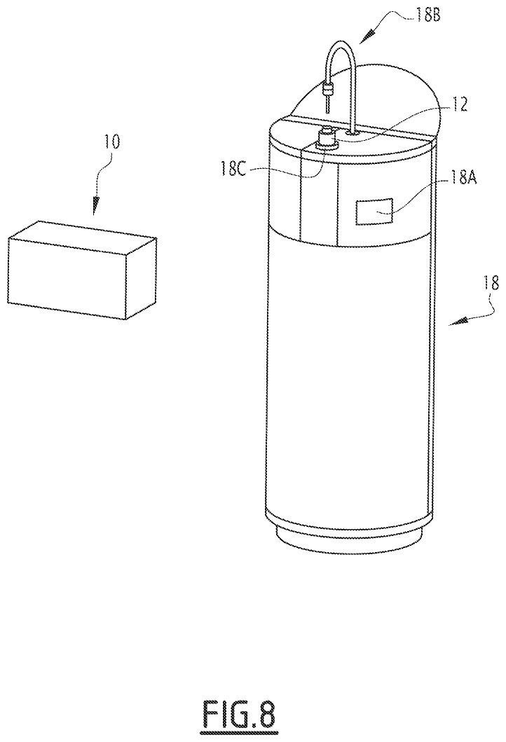

FIG. 8 is a schematic perspective view of a decontamination device according to the invention, associated with a product dispensing machine, but separate from the product dispensing machine;

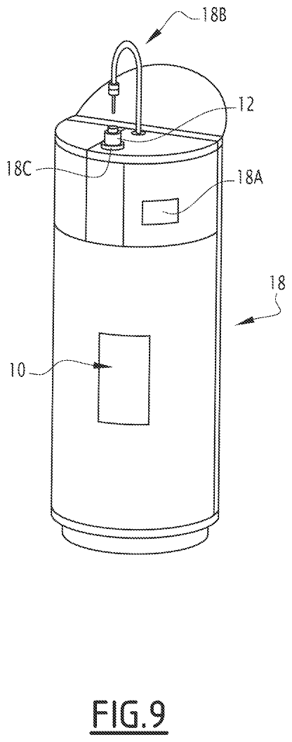

FIG. 9 is a view similar to FIG. 8 of a decontamination device according to the invention, integrated within a product distribution machine.

A first decontamination device 10 according to the invention is illustrated schematically in FIGS. 1 and 2.

In particular, the device 10 is intended to decontaminate a hollow object 12 comprising an internal cavity 14 opening out through an opening 16.

Decontamination of the hollow object 12 reduces the presence of viable microorganisms, such as germs. Decontamination is, for example, a sterilization of at least the internal cavity 14 of the hollow object 12. A sterilization is, for example, a treatment to reduce the bacterial load, and in particular to obtain a theoretical probability of the presence of a viable microorganism less than or equal to 10-6 cfu.

The decontamination advantageously induces a reduction of at least 1 log, preferably at least 2 log, on a 1000 CFU/vial Aspergillus seeding

The hollow object 12 is intended, after decontamination in the device 10, to receive a product into the internal cavity 14 through the opening 16.

The product is delivered by a bulk product dispensing machine 18, examples of which are shown in FIGS. 8 and 9. The machine comprises in a known manner a product reservoir 18A, a product dispensing nozzle 18B, for introducing the product into the hollow object 12, and optionally a measuring assembly 18C for measuring the amount of product introduced into the cavity 14 of the hollow object 12. The hollow object 12, after decontamination, is able to be placed facing the dispensing nozzle 18B to receive a dose of product.

In the example of FIG. 8, the decontamination device 10 is a stand-alone device advantageously associated with a separate machine 18 for dispensing bulk product into the hollow object 12. In the example shown in FIG. 9, the decontamination device 10 is integrated within a machine 18 for dispensing bulk material into the hollow object 12.

The machine is for example such as described in the French patent application No. FR2008422.

The product is for example solid, powdery or liquid, preferably liquid. It can be an anhydrous or aqueous composition, or a single or multiple emulsion/dispersion, for example as described in WO2010063937, WO2018167309, WO2012120043, WO2017046305 or in the French patent applications filed under No. FR2005408 and FR2005410.

Advantageously, the product is a food product, a cleaning product (especially a detergent), or a cosmetic product, preferably a cosmetic product.

Advantageously, the hollow object 12 presenting a cavity 14 is a package intended to receive the product.

The packaging is, in particular, a cosmetic packaging or a bottle.

This packaging can be of any material and of any size.

The or each material forming the packaging is adapted to the decontamination technology used to be resistant to the decontamination vector used.

For example, a package according to the invention is formed:
from glass;
from plastic, for example polyethylene (PE), ethylene glycol terephthalate (PETG), polyethylene terephthalate (PET), styrene-acrylonitrile (SAN or ABS), polypropylene (PP), polyvinyl chloride (PVC) or polystyrene (PS); or
metal, for example, aluminum,
or even a combination of the above materials.

The configuration, size, and/or volume (at least internally) of the package are adapted to the decontamination/sterilization technology used, and in the case of a technology based on light from a light-emitting diode (hereinafter LED) or ultraviolet light (hereinafter UV), to the size of the light source generating the light and in particular to its diameter.

For example, a package intended, according to the invention, presents a capacity between 5 ml and 1000 ml, preferably between 10 ml and 750 ml, in particular between 30 ml and 500 ml, and better still, between 50 ml and 250 ml.

In this example, the hollow object 12 includes a container 20 defining the internal cavity 14, and a neck 22 defining the access opening 16 to the internal cavity 14. The internal cavity 14 preferably extends along a longitudinal axis A-A′ through the opening 16.

With reference to FIGS. 1 and 2, the device 10 includes a decontamination unit 24, a protective enclosure 26, at least partially receiving the decontamination unit 24, and advantageously, a sensor system 28. The device 10 further includes a control unit 30, able to control at least the decontamination unit 24.

The enclosure 26 includes a case 27 defining an inner space 27A intended to contain the decontamination unit 24 during a decontamination sequence. The enclosure 27 includes partitions that are advantageously opaque and defines at least one passage 27B for access to the inner space 27A, intended for placing the hollow object 12 in the decontamination unit 24.

The enclosure 26 further includes a closure wall 29, movable relative to the access passage 27B between an open configuration of the access passage 27B, visible in FIG. 1, allowing the hollow object 12 to be inserted into the decontamination unit 24 and a closed configuration of the access passage 27B, visible in FIG. 2, preventing access to the decontamination unit 24.

The closure wall 29 is movable in translation and/or rotation relative to the access passage 27B between the open configuration and the closed configuration.

In the example shown in FIGS. 1 and 2, the closure wall 29 is a panel that is movably mounted, preferably in translation, on the housing 27.

As illustrated in FIG. 2, the decontamination unit 24 includes a holder 32, a decontamination member 34, and a mechanism 36 for relative displacement between the holder 32 and the decontamination member 34, between a rest position intended for loading the hollow object 12 into the holder 32 (visible in FIG. 1) and a decontamination position, in which the decontamination member 34 is inserted into the cavity 14 of the hollow object 12 (visible in FIG. 2).

The holder 32 is able to position and hold the hollow object 12 in the device 10, avoiding the introduction of a hollow object 12 unsuitable for the enclosure 26 and the decontamination unit 24, in particular the decontamination member 34.

In this example, the holder 32 includes a shell 40 defining at least one impression 42 of a shape complementary to at least a portion of the hollow object 12, and advantageously a jumper 44 for receiving the neck 22.

Advantageously, the holder 32 includes a mobile flap 46 for locking the positioning of the hollow object 12 in the holder 32, which is movably mounted on the shell 40.

The shell 40 is advantageously a shaped part, in particular made of metal, plastic or resin, for example using 3D printing technology.

Thus, the holder 32 is configured to provide coaxiality between the internal cavity 14 of the hollow object 12, in particular the axis A-A′ of the access opening 16 of the hollow object 12, and the axis B-B′ of relative movement of the decontamination member 34 with respect to the holder 32.

As will be seen below, the holder 32 is also configured to provide smooth closure of the enclosure 26, and to allow relative movement between the decontamination member 34, the holder 32, and the hollow object 12, when the hollow object 12 is received in the holder 32.

The indentation 42 is formed in the shell 40. This presents a shape complementary to at least one region of the container 20.

In one alternative, the holder 32 defines a plurality of indentations 42 suitable for hollow objects 12 of different shapes and/or sizes.

The jumper 44 is intended to receive the neck 22. It also presents a shape complementary to at least one outer surface of the neck 22.

The indentation 42 and the jumper 44 define the position along the axis of movement B-B′ and the orientation of the hollow object 12 when the hollow object 12 is received in the holder 32. They form a guiding and/or receiving region for the hollow object 12 in the holder 32.

Eventually, the indentation 42 and/or the jumper 44 present at least one angular indexing relief (not shown), allowing the hollow object 12 to be oriented about its longitudinal axis A-A′ according to a predefined orientation.

The flap 46 is movably mounted on the shell 40 between a position for placing the hollow object 12 in the holder 32, visible in FIG. 1, and a position for holding the hollow object 12 in place in the holder 32, visible in FIG. 2.

In this example, the flap 46 is hinged to the shell 40 and is pivotable relative to the shell 40 between the placement position and the holding position. Alternatively, not shown, the flap 46 is movable in translation relative to the shell 40.

Preferably, the holder 32 includes a locking element (not shown) for the flap 46 in the holding position. This locking element is, for example, operated manually by a user of the device 10, or by an actuator controlled by the control unit 30, in particular an electromechanical actuator.

In an alternative, not shown, the holder 32 is devoid of a flap 46. The cavity 42 then presents a shape that is suitable and sufficient to prevent any displacement of the object 12 placed in the holder 32, particularly during a relative displacement between the holder 32 and the decontamination member 34.

The decontamination member 34 is chosen according to the decontamination method implemented, which may be any method known to the man of the trade.

The shape and dimensions of the decontamination member 34 are chosen to allow its insertion into the internal cavity 14, through the opening 16, depending on the dimensions and/or shape of the internal cavity 14 and if necessary the opening 16.

Preferably, the decontamination member 34 presents a length selected to remain clear of the bottom of the internal cavity 14.

In one example, the decontamination member 34 is able to emit vapor into the internal cavity 14, in particular dry vapor, to diffuse ozone into the internal cavity 14 (see for example EP0436042A1, US20060032189A1) and/or to spray a disinfecting and rinsing solution.

Alternatively or additionally, the decontamination member 34 is able to emit light, for example LED light, UV light (see for example EP2816002), and/or emitting electromagnetic radiation (see for example US2007258851, U.S. Pat. No. 6,085,492).

Preferably, the decontamination member 34 is based on an anhydrous decontamination technique.

Advantageously, the decontamination member 34 is based on a decontamination technique of the LED or UV light type, advantageously in the form of pulsed light, and more preferably UV light. A UV light is preferably a light having an emission peak with a wavelength below 380 nm, advantageously between 10 nm and 380 nm.

The decontamination member 34 then includes a light source 50, in particular a lamp, preferably made of quartz, of the medium-pressure xenon type operating, optionally in pulsed mode, and optionally provided with a radio-frequency reflector to also decontaminate the neck 22 of the hollow object 12. It also includes a power supply 52 for the light source 50, able to be controlled by the control unit 30.

The light source 50, is intended to be positioned in the cavity 14, such that a portion of the radiating body of this light source 50 protrudes from the cavity of the hollow object 12.

Preferably, the light source 50, presents an elongated shape according to an axis B-B′ of displacement of the decontamination member 34 relative to the holder 32, in particular a cylindrical or oblong shape.

The light source 50, can also contribute to the disinfection by a thermal effect and/or a black body effect.

A decontamination member 34, is in particular as described in WO2010012915.

The decontamination member 34, is movable relative to the holder 32 via the displacement mechanism 36, between the rest position intended for positioning the hollow object 12 in the holder 32 and the decontamination position, in which at least a portion of the decontamination member 34 is intended to be inserted into the cavity 14 of the hollow object 12.

This displacement is advantageously a translation along the B-B′ axis of the decontamination member 34.

In the example shown in FIGS. 1 and 2, the holder 32 is movable in the enclosure 26 and the decontamination member 34 is static relative to the enclosure 26. The displacement mechanism 36 is able to generate a displacement of the holder 32 with respect to the decontamination member 34.

In an alternative, not shown, the holder 32 is static relative to the enclosure 26 and the decontamination member 34 is movable in the enclosure 26. The displacement mechanism 36 is able to generate a displacement of the decontamination member 34 relative to the holder 36.

In yet another alternative, not shown, the holder 32 and the decontamination member 34 are movable within the enclosure 26. The displacement mechanism 36 is able to cause simultaneous movement of the decontamination member 34 and the holder 36 toward or away from each other.

In this example, the displacement mechanism 36 includes an actuator 60, for example electromechanical, intended to be controlled by the control unit 30 or by a control button operated by the user, to cause the relative displacement between the holder 36 and the decontamination member 34.

Advantageously, the displacement mechanism 36 includes a guide member 62, on which the holder 32 and/or the decontamination member 34 is intended to slide during the displacement between the rest position and the decontamination position. This prevents any mishandling of the displacement mechanism 36.

Alternatively, the displacement mechanism 36 includes a manual displacement member, instead of the actuator 60, for example a handle or a lever. The relative displacement between the holder 32 and the decontamination member 34 is then performed manually by the user of the device 10, which simplifies the movement mechanism 36 and ensures its robustness.

In the rest position, as illustrated in FIG. 1, the holder 32 is relatively far from the decontamination member 34 along the axis of displacement B-B′. The closure wall 29 preferably occupies its open configuration.

The holder 32 is arranged facing the access passage 27B, the decontamination member 34 preferably being arranged away from the access passage 27B by being masked by a partition of the enclosure 26.

This position allows the hollow object 12 to be placed in the holder 32, limiting the risk of contact with the decontamination member 34.

In one alternative, in the rest position, the holder 32 protrudes from the enclosure 26 through the access passage 27B.

In the decontamination position, the decontamination member 34 and the holder have moved closer to each other along the axis B-B′, so that the decontamination member 34 is positioned opposite the holder 32, and is inserted into the inner cavity 14 of the hollow object 12, preferably without contact with the hollow object 12, at least with the neck 22 and/or the bottom of the hollow object 12.

The closure wall 29 then occupies its closed configuration, preventing access to the inner space 27A of the housing 27 through the access passage 27B and opaquely sealing the housing 27.

Advantageously, the relative displacement of the decontamination member 34 relative to the access passage 27B, from the rest position to the decontamination position, causes the relative displacement of the closure wall 29 relative to the holder 32, from the open configuration to the closed configuration. Advantageously, the relative displacement of the closure wall 29 relative to the access passage 27B from the open configuration to the closed configuration causes the relative displacement of the decontamination member 34 relative to the holder 32 from the rest position to the decontamination position.

Preferably, the displacement mechanism 36 includes an assembly for coupling the decontamination member 34 and/or the holder 32 to the closure wall 29 to subject the relative displacement of the closure wall 29 relative to the access passage 27B to the relative displacement between the decontamination member 34 and the holder 32.

When the holder 32 is movable, the decontamination member 34 being static, the coupling assembly subjects the relative displacement of the closure wall 29 relative to the passage 27B to the displacement of the holder 32.

When the holder 32 is static, the decontamination member 34 being movable, the coupling assembly subjects the relative displacement of the closure wall 29 relative to the access passage 27B to the displacement of the decontamination member 34.

In one alternative, the closure wall 29 is maintained permanently in the closed configuration, including in the rest position, for example by being spring biased. In this case, the insertion of the hollow object 12 into the holder 32 requires the user or the control unit to first move the closure wall 29 into the open configuration, with the holder 32 in the rest position.

More generally, all displacements are generated manually by the user or alternatively, by the control unit 30.

The system of sensors 28 includes a first sensor 70 for detecting the placement of the hollow object 12 in the holder 32, a second sensor 72 for detecting the closed configuration of the closure wall 29, and/or a third sensor 74 for detecting the decontamination position of the decontamination member 34. Advantageously, it includes a fourth sensor 76 for detecting the position of the flap 46.

The sensors 70 to 74 are connected to the control unit 30.

The first sensor 70 is able to identify the presence of a hollow object 12 placed in the holder 32, and thus to allow the control unit 30 to authorize or not the activation of the decontamination process described below.

Advantageously, the first sensor 70 is also able to identify the shape and/or weight of the hollow object 12 positioned on the holder 32, in particular so as to adjust the relative displacement of the decontamination member 34 relative to the holder 32 and/or the decontamination sequence.

Preferably, it is able to detect an incorrect positioning of the hollow object 12 relative to the decontamination member 34 to allow the control unit 30 to prevent the relative displacement between the holder 32 and the decontamination member 34 and any decontamination sequence.

Advantageously, the sensor 70 may be coupled to at least one light indicator visible to the operator or consumer informing of the presence of a hollow object 12 suitable for and properly placed in the holder 32. Alternatively, the sensor 70 may be coupled to at least two light indicators, preferably of different colors, visible to the operator or consumer, the first indicator informing of the absence of a hollow object 12 in the holder 32 or of the presence of a hollow object 12 not suitable for or not correctly placed in the holder 32 and the second light indicator informing of the presence of a hollow object 12 suitable for and correctly placed in the holder 32.

The second sensor 72 is preferably able to detect the closed configuration and the open configuration of the closure wall 29, to allow the control unit 30 to respectively authorize or prevent the activation of the decontamination member 34 and/or the relative displacement of the holder 32 relative to the decontamination member 34.

The third sensor 74 is advantageously able to detect at least the rest position and the decontamination position.

The fourth sensor 76 is able to detect the holding position of the flap 46 to advantageously allow the control unit 30 to lock the flap 46 in position, in this position.

The control unit 30 includes at least one processor and a memory containing software modules that can be executed by the processor. It includes an interface able to communicate with the or each sensor 70 to 76 of the sensor system 28 and in particular able to detect the presence of the hollow object 12 on the holder 32, and advantageously its size and/or its weight and/or its appropriate positioning.

It includes a module for selectively activating the relative displacement between the holder 32 and the decontamination member 34 toward the decontamination position, and a module for activating the decontamination member 34 in order to generate a decontamination, on the basis of the data acquired by the sensors 70 to 76.

Thus, the control unit 30 is able to activate the decontamination member 34. By “activating the decontamination member”, is meant that the control unit 30 is able to at least make the decontamination member 34 active in the implementation of a decontamination treatment, for example by switching on the light source 50, by starting the distribution of vapor, gas or disinfectant and rinsing solution by the decontamination member 34.

Possibly, as will be seen below, the activation of the decontamination member 34 is accompanied or preceded by the control of the relative displacement of the decontamination member 34 relative to the holder 32 from the rest position to the decontamination position.

Advantageously, the control unit 30 comprises at least one module for detecting problems with the closure wall 29, the holder 32, the displacement mechanism 36 and/or the decontamination member 34 and/or optionally the flap 46.

Advantageously, in the absence of a hollow object 12 carried by the holder 32, the control unit 30 is configured to control an automatic return to a standby configuration, in which the closure wall 29 is in the closed configuration. In this case, the decontamination member 34 optionally occupies the decontamination position relative to the holder 32, without being activated.

Preferably, in particular in the device 10 shown in FIGS. 1 and 2, the return to the standby configuration is controlled automatically after a time delay of between 5 seconds and 30 seconds, in particular between 10 seconds and 20 seconds, and better between 10 seconds and 15 seconds.

According to a first embodiment, the control unit 30 comprises at least one physical or software button able to be pressed by the user to initiate the decontamination process described later.

According to one alternative, the control unit 30 comprises at least two physical or software buttons, where:
the first button is able to ensure the relative displacement of the closure wall 29 relative to the access passage 27B between the open configuration and the closed configuration, or even the relative displacement of the decontamination member 34 relative to the holder between the rest position and the decontamination position, and
the second button is able to be pressed by a user to ensure at least the activation of the decontamination member 34.

Preferably, the housing may further include at least one emergency stop button.

A method for decontaminating a hollow object 12, implemented using the decontamination device 10 according to the invention, will now be described.

Initially, the decontamination device 10 is provided in the rest position of the decontamination member 30 relative to the holder 32 and in the open configuration of the closure wall 29 relative to the access passage 27B.

According to one alternative, the closure wall 29 has previously been moved from a closed configuration to an open configuration relative to the access passage 27B and the decontamination member 34 has been moved from a decontamination position to a rest position relative to the holder 32.

A hollow object 12 presenting a cavity 14 is then inserted into the holder 32 by the user. When present, the flap 46 is initially placed in the placement position, then is operated into the holding position after the hollow object has been placed in the holder 32. The flap 46 is preferably locked in the holding position.

A decontamination sequence is then implemented.

The closure wall 29 transitions into the closed configuration, and the displacement mechanism 36 moves the decontamination member 34 closer to the holder 32 to transition from the rest position to the decontamination position in which the decontamination member 34 is inserted into the cavity 14 of the hollow object 12.

When the closure wall 29 is in the closed configuration, at least the holder 32, the hollow object 12, and the decontamination member 34 are completely contained within the inner space 27A of the enclosure 26, closed by the closure wall 29.

Optionally, the transition of the closure wall 29 from the open configuration to the closed configuration and the approach of the decontamination member 34 to the holder 32 to transition from the rest position to the decontamination position are simultaneous.

Then, the decontamination member 34 is activated by the control unit 30, based on the measurements received from at least one sensor 70 to 74 of the system of sensors 28.

Optionally, the activation of the decontamination member 34 is initiated during the transition from the rest position to the decontamination position, but only after the closed configuration has been reached.

In one embodiment, the decontamination member 34 is activated only once, either over a very short time (as a flash) or continuously.

Alternatively, the decontamination member 34 is activated at least twice, preferably at least three times, etc., preferably in the form of successive flashes. This prevents the decontamination member 34 from heating up, and thus from being damaged, and is less energy intensive. The decontamination is more efficient, and reduces the risk of altering the screen printing that may be present on the hollow object 12.

Advantageously, the decontamination member 34 is activated during its introduction into the cavity 14, so as to shorten the duration of a decontamination sequence and therefore the duration of the decontamination process and to improve the efficiency of the decontamination of the hollow object 12.

Then, the decontamination member 34 is deactivated. The displacement mechanism 36 moves the decontamination member 34 away from the holder 32, from the decontamination position to the rest position. The decontamination member 34 exits the cavity 14.

Deactivation of the decontamination member 34 may occur during the displacement from the decontamination position to the rest position.

The closure wall 29 moves relative to the access passage 27B to transition from the closed configuration to the open configuration.

In one embodiment, not shown, when the holder 32 is movable relative to the enclosure 26, in the rest position, the holder 32 advantageously at least partially protrudes from the enclosure 26.

Optionally, the transition from the decontamination position to the rest position is simultaneous with the transition from the closed configuration to the open configuration, but occurs only after the deactivation of the decontamination member 34.

Then, when the holder comprises a flap 46, this is unlocked and transitions to its placement position.

The decontaminated hollow object 12 is then extracted from the holder 32 and retrieved by the user, in order to receive a dose of product dispensed by the machine 18.

Eventually, the control unit 30 controls an automatic return to a standby configuration, in which the closure wall 29 is in the closed configuration. Eventually, the decontamination member 34 occupies the decontamination position relative to the holder 32, without being activated.

The process can then be implemented for a new hollow object 12, by repeating the above steps.

Thanks to the device 10 and the method according to the invention, it is possible to ensure in a simple and secure manner, for example in a store or on a demonstration site (in particular a trade fair), a satisfactory decontamination of a hollow object 12 presenting a cavity 14 (in particular a package) for subsequent filling.

Furthermore, any interaction between the hollow object 12 and the decontamination member 34 is limited or even prevented, thus avoiding or minimizing the risk of contamination and/or breakage of the decontamination member 34.

The device 10 according to the invention is therefore particularly suitable for the one-time implementation of a decontamination of a hollow object 12, in contrast to a continuously operating industrial decontamination plant.

The decontamination device 10 illustrated in FIG. 3 differs from that shown in FIG. 1 in that the housing 27 is a drawer that is displaceable relative to a frame 80 that contains the closure wall 29. Advantageously, the closure wall 29 is static relative to the frame 80.

Advantageously, the frame 80 is that of a product dispensing machine 18, such as illustrated in FIG. 9.

When the drawer is extracted from the frame 80, the access passage 27B transitions to an open configuration relative to the closure wall 29. It returns to a closed configuration when the drawer is inserted into the frame 80.

In the example represented in FIG. 3, the holder 32 and the decontamination member 34 are displaced in conjunction with the drawer as the drawer is withdrawn from the frame 80. Alternatively, only the holder 32 or only the decontamination member 34 is displaced in conjunction with the drawer, the opening of the drawer causing the decontamination member 34 to transition into the resting position relative to the holder 32, the closing of the drawer causing the decontamination member 34 to transition into the decontamination position relative to the holder 32.

The operation of the decontamination device 10 illustrated in FIG. 3 is otherwise similar to that illustrated in FIG. 1.

In the alternative illustrated in FIGS. 4 to 6, the device 10 includes an enclosure 26 presenting a partition 81, advantageously vertical, defining the access passage 27B opening into the inner space 27A.

The inner space 27A of the enclosure 26 comprises a region 82A for access to the holder 32 opening out to the outside of the device 10 via the access passage 27B, and a technical region 82B, advantageously separated from the access region 82A by at least one partition 82C internal to the inner space, here a horizontal partition.

The access region 82A is closed inwardly by a cover 83, arranged opposite the access passage 27B. Thus, the access region 82A and access passage 27B define an alcove provided in the enclosure 26.

In this example, the access region 82A is located above the technical region 82B.

The holder 32 is contained within the access region 82A. It is static here, particularly relative to the partition 81. When the hollow object 12 is mounted on the holder 32, it is fully contained within the access region 82A.

With reference to FIG. 6, the holder 32 includes a sleeve 90 for positioning the neck 22 of the hollow object 12, complementary to at least the end of the neck 22 of the hollow object 12, for example a neck of the hollow object 12. The sleeve 90 forms a region for guiding and positioning the hollow object 12.

Preferably, the neck 22 screws onto the sleeve 90. The sleeve 90 then defines an internal thread complementary to an external thread on the neck 22 of the hollow object 12.

Upon insertion of the hollow object 12 into the holder 32, the hollow object 12 is positioned within the sleeve 90 until the lip of the neck 22 reaches a stop 91 which actuates the first presence sensor 70, here inserted below the stop 91.

The sleeve 90 linearly guides the hollow object 20 and thus ensures that the decontamination member 34 is reliably introduced into the cavity 14, to its maximum extent. This ensures the most satisfactory guidance and thus the most satisfactory coaxiality with the decontamination member 34.

The decontamination member 34 is mounted so as to be movable relative to the holder 32, in particular so as to be movable in translation along a vertical axis B-B′, between the rest position and the decontamination position.

In the rest position, the decontamination member 34 is here completely contained in the technical region 82B, away from the access region 82A and the access passage 27B.

In the decontamination position, the decontamination member 34 is at least partially inserted into the access region 82A, so as to enter the internal cavity 14 of the hollow object 12 mounted on the holder 32. In particular, the decontamination member 34 protrudes through the sleeve 90 to reach the internal cavity 14.

Here, the closure wall 29 is a panel movably mounted relative to the access passage 27B between the open configuration and the closed configuration.

The displacement of the closure wall 29 is here coupled with that of the decontamination member 34.

Thus, preferably, the closure wall 29 is movable in translation parallel to the displacement axis B-B′ (here vertical), between the open configuration, visible in FIG. 4 and the closed configuration, visible in FIG. 5.

In the open configuration, the closure wall 29 is at least partially retracted (here downward), to at least partially free the access passage 27B and allow a user to load or unload a hollow object 12 into the holder 32. The alcove is then accessible.

In the closed configuration, the closure wall 29 closes the entire access passage 27B, to prevent a user from loading or unloading a hollow object 12 into the holder 32 and advantageously to protect it from any light or any product projection from the decontamination member 34. The alcove is then inaccessible.

In addition, the holder 32 is advantageously arranged vertically above the decontamination member 34, the hollow object 12 then being placed with the internal cavity 14 opening downwards. This prevents residues from entering the cavity 14 if the decontamination member 34 breaks, for example due to an impact.

To ensure precise movement of the decontamination member 34 relative to the holder 32, and to maintain alignment of the decontamination member 34 relative to the hollow object 12 mounted in the holder 32, the actuator 60 includes a movable carriage 84 slidably mounted on at least one guide rod 62, to carry the decontamination member 34.

The movable carriage 84 includes two lateral brackets 84A, arranged parallel to each other on either side of the displacement axis B-B′. The decontamination member 34, in particular the generator 52, is clamped between the brackets 84A.

The device 10 illustrated in FIGS. 4 to 6 is advantageously a self-contained device, for example a terminal, the partition 81 forming an outer surface of the terminal.

Alternatively, the device 10 illustrated by FIGS. 4 to 6 is integrated within a machine 18 for dispensing bulk material into the hollow object 12, the partition 81 preferably forming an outer wall of the machine 18.

In an alternative illustrated in FIG. 7, the holder 32 includes a guiding and positioning region adapted to position and hold the hollow object 12 by simple gravity, and in particular by guiding at least the neck 22 by sliding over the guiding and positioning region.

The guiding and positioning region includes a downwardly converging surface of revolution 92, for example a frustoconical surface.

In this alternative, the decontamination unit 24 is advantageously positioned according to a vertical axis. The holder 32 is configured to receive the object 12 upside down. It is placed above the decontamination member 34.

This alternative is advantageous in that it avoids forcing coaxiality between the hollow object 12 and the decontamination member 34, by allowing a displacement clearance between the hollow object 12 and the decontamination member 34, even in the presence of very small spacings between the decontamination member 34 and the neck 22 of the hollow object 12.

In one alternative (shown in dotted line in FIG. 7), the decontamination unit 24 advantageously comprises an outer envelope 100 coaxial with the decontamination member 34. The envelope 100 presents a funnel-shaped open end at the free end of the decontamination member 34 intended to enter the cavity 14 of the hollow object 12. The end of the envelope 100 is configured to penetrate at least a portion of the neck 22 of the hollow object 12 to allow the transition of the decontamination member 34. The envelope 100 thus ensures dual guidance of the decontamination member 34 into the hollow object 12 and prevents, or even effectively prevents, any risk of contact or even collision between the decontamination member 34 and the neck 22 of the hollow object 12.

1-16. (canceled)

17. A device for decontaminating a hollow object defining an internal cavity, including:
a decontamination unit, the decontamination unit including a holder intended to receive and position the hollow object, a decontamination member intended to be introduced into the internal cavity, and a mechanism for the relative displacement of the decontamination member relative to the holder able to displace the decontamination member and the holder relatively the one in relation to the other between a rest position intended for positioning the hollow object in the holder and a decontamination position, in which the decontamination member is intended to be inserted into the internal cavity of the hollow object received in the holder
a control unit able to activate the decontamination member in the decontamination position,
wherein:
a protective enclosure defines an inner space containing at least a portion of the decontamination member and the holder at least in the decontamination position, the enclosure defining an access passage to the inner space and at least one wall for obturating the access passage the closure wall and the access passage being movable, the one relative to the other, between an open configuration of the access passage which the closure wall is able to occupy in the rest position of the decontamination member and a closed configuration of the access passage which the closure wall occupies in the decontamination position of the decontamination member.

18. The device according to claim 17, wherein the relative displacement of the decontamination member relative to the access passage from the rest position to the decontamination position causes relative displacement of the closure wall relative to the holder from the open configuration to the closed configuration,
and/or wherein the relative displacement of the closure wall relative to the access passage from the open configuration to the closed configuration causes the relative displacement of the decontamination member relative to the holder from the rest position to the decontamination position

19. The device according to claim 17, wherein the relative displacement of the decontamination member relative to the holder and the relative displacement of the closure wall relative to the access passage is coupled the one to the other.

20. The device according to claim 17 wherein, in the rest position, the closure wall is displaceable relative to the access passage between the open configuration and the closed configuration.

21. The device according to claim 20, wherein the closure wall is held at rest in the closed configuration.

22. The device according to claim 17, wherein the control unit is able to control the relative displacement of the decontamination member relative to the holder between the rest position and the decontamination position and/or is able to control the relative displacement of the closure wall relative to the access passage from the open configuration to the closed configuration.

23. The device according to claim 17 including a first sensor for detecting the placement of the hollow object in the holder and/or a second sensor for detecting the closed configuration of the closure wall, and/or a third sensor for detecting the decontamination position, the control unit being able to activate the decontamination member when the first sensor detects that the object is placed in the holder and/or when the second sensor detects that the closure wall is in the closed configuration, and/or when the third sensor detects that the decontamination member is in the decontamination position.

24. The device according to claim 17, wherein the closure wall is a panel able to be displaced in translation or rotation relative to the access passage or wherein the enclosure comprises a movable drawer defining the access passage, the drawer being movable relative to the closure wall.

25. The device according to claim 17, wherein the holder includes a region for guiding and/or receiving at least a portion of the hollow object, in particular a neck of the hollow object, able to fix the position and alignment of the hollow object in the holder, the guiding and/or receiving region being, in particular, a sleeve complementary to a neck of the hollow object, a surface for guiding a neck of the hollow object by gravity or an indentation for receiving the hollow object, the shape of which is complementary to that of the hollow object and which is able to fix the position and the alignment of the hollow object in the holder.

26. The device according to claim 25, wherein the holder includes a flap for holding the hollow object in the guiding and/or receiving region, displaceable, in particular pivotable, between a position for placing the hollow object in the guiding and/or receiving region and a position for holding the hollow object in the guiding and/or receiving region.

27. The device according to claim 17, wherein the decontamination member comprises a light source, in particular a continuous or pulsed UV light emission source.

28. The device according to claim 17, wherein the holder is arranged above the decontamination member in the rest position.

29. A machine for dispensing bulk product into a hollow object defining an internal cavity, the machine comprising at least one product reservoir, at least one dispensing nozzle intended to be placed facing the internal cavity of the hollow object and optionally an assembly for measuring the quantity of product introduced into the internal cavity of the hollow object, the machine further including a decontamination device according to claim 17.

30. A method for decontaminating a hollow object presenting an internal cavity comprising the following steps:
providing a decontamination device according to claim 17;
implementing a decontamination sequence comprising the following steps:
the decontamination member being in its rest position and the closure wall being in its open configuration, introducing a hollow object presenting an internal cavity into the holder
transition of the closure wall to the closed configuration and transition of the decontamination member to the decontamination position, the decontamination member being inserted into the internal cavity;
activation of the decontamination member by the control unit; and, at the end of the decontamination sequence:
transition of the closure wall into the open configuration and transition of the decontamination member into the rest position, the decontamination member exiting the internal cavity;
removing the hollow object from the holder.

31. The method according to claim 30, wherein activation of the decontamination member occurs after the transition of the closure wall into the closed configuration and before the decontamination member reaches its decontamination position.

32. The method according to claim 30, wherein, after the removal of the hollow object from the holder, the control unit controls the transition of the closure wall into the closed configuration and/or the transition of the displacement member from the rest position to the decontamination position, without activating the decontamination member.

33. The method according to claim 32, further including the following steps:
providing a new hollow object to be decontaminated;
transition of the decontamination member to its rest position, and/or the closure wall to its open configuration,
implementation of a new decontamination sequence comprising the following steps:
introducing the new hollow object presenting an internal cavity into the holder,
transition of the closure wall into the closed configuration and transition of the decontamination member into its decontamination position, the decontamination member being inserted into the internal cavity of the new hollow object
activation of the decontamination member by the control unit.
